(19) United States
(12) Patent Application Publication
(10) Pub. No.: US 2007/0179364 A1
(43) Pub. Date: Aug. 2, 2007

(54) Method of classifying gerd

(75) Inventors: Bernd Pfaffenberger, Konstanz (DE); Gudrun Gatz, Allensbach (DE)

Correspondence Address: NATH & ASSOCIATES PLLC, 112 South West Street, Alexandria, VA, 22314, US

(73) Assignee: ALTANA PHARMA AG, KONSTANZ (DE)

(21) Appl. No.: 11/587,274

(22) PCT Filed: Apr. 29, 2005
(86) PCT No.: PCT/EP05/04658

§ 371 Date: Nov. 16, 2006

(30) Foreign Application Priority Data
Apr. 30, 2004 (EP) 04010347.5

(51) Int. Cl. A61B 5/00

(52) U.S. Cl. 600/309

(57) ABSTRACT

The present invention relates to a method of diagnosing GERD (gastroesophageal reflux disease) in a human subject suspected to have GERD which comprises grading the endoscopic status and the symptomatic status of the human subject with individual grading parameters, and combining said grading parameters into a single parameter. The present invention also relates to a processing device for use in the method according to the invention and a means for displaying the single parameter. Furthermore the invention is concerned with uses of active ingredients having an ameliorative effect for GERD for the preparation of a medicament for treating GERD in a human patient who has been diagnosed GERD-positive by the method of the invention. The method of the invention represents the first integrated concept of diagnosing GERD.

### FIELD OF THE INVENTION

The present invention relates to a method of diagnosing GERD, and a processing device and a displaying means used in this method, as well as a method of treating GERD using the above method.

### RELATED PRIOR ART

GERD (gastroesophageal reflux disease) is a common and highly prevalent condition characterized by the typical symptoms of heartburn, acid regurgitation, and pain on swallowing. Other associated reflux symptoms are e.g., burning in the throat, dysphagia, pressure in the upper and lower abdomen, sore throat, nausea or vomiting. The term GERD is generally understood to comprise both, erosive esophagitis and endoscopy negative reflux disease (enGERD). GERD affects all individuals who are exposed to the risk of physical complications from gastro-esophageal reflux or who experience clinically significant impairment of health-related well being (quality of life) due to reflux-related symptoms, after adequate reassurance of the benign nature of their symptoms.

So far, GERD has been diagnosed in two ways, namely endoscopically and symptomatically. More specifically, the symptomatic diagnosis has gained especially in importance when diagnostic findings were negative. This meant in practice that the symptomatic level of diagnosis was usually only entered when the endoscopic findings were negative. The above separate and independent usage of endoscopic and symptomatic aspects of diagnosing GERD has been applied even though these aspects were known not to be correlated. That is, endoscopically healed patients can still suffer from symptoms and patients with symptoms do not necessarily have positive endoscopic result. In terms of treating GERD, medications administered apparently have a healing effect on the esophageal mucosa, i.e. are effective to heal GERD on the endoscopic level, but many of these patients continue to be symptomatic.

In order to assess the endoscopic level of GERD, the so-called LA-classification described in Gut 1999; 45: 172-180, has been applied most often. The LA-classification has been extensively validated and is therefore internationally accepted and recommended. It is based on an observation of mucosal breaks in esophageal endoscopy. The four grades of severity A to D are based on the extent of visible mucosal breaks. Recently, P. J. Kahrilas has adapted the LA-classification by adding the “not-present” grade (P. J. Kahrilas et al., Aliment Pharmacol Ther. 2000; Vol. 14 (10): 1249-58). Grade 0 of the above adapted LA-classification corresponds to endoscopy negative reflux disease (enGERD).

In order to evaluate the symptomatic level of GERD, which has usually been carried out only after endoscopic healing, either an investigator's assessment or a self-assessment of the patient with the aid of questionnaires has been performed.

Despite the known lacking correlation of the endoscopic and symptomatic level of diagnosing GERD, no attempt has been made so far to resolve the inconsistencies caused by the lack of correlation. Therefore, a “united” concept of diagnosing and healing GERD was in high demand.

More specifically, the object underlying the present invention resides in an integrated concept of diagnosing GERD and classifying the disease status thereof allowing the exact categorization of individuals having or being suspected to have GERD, which can enable the optimum GERD therapy to be applied.

### SUMMARY OF THE INVENTION

The above objective problem has been solved by a method of diagnosing GERD in a human subject suspected to have GERD which method comprises grading the endoscopic status and the symptomatic status of the human subject with individual grading parameters, and combining these grading parameters into a single parameter. Moreover, the present invention relates to a processing device comprising means for diagnosing GERD.

According to another aspect, the present invention relates to a method of treating GERD in a human patient by administering, to a patient, a medicament having an ameliorative effect for GERD containing an active ingredient which may be selected from the class of proton pump inhibitors, H₂ receptor antagonists, and acid pump antagonists, wherein the therapeutic need of the patient is defined by the method involving a single parameter according to the present invention. Furthermore, the present invention pertains to a means for displaying the single parameter.

The method of diagnosing GERD according to the invention by way of assessment of the endoscopic and symptomatic level at the same point of time allows to follow the progress of GERD healing and to assess the success of therapy, more effectively than the separate and independent assessment of the endoscopic and symptomatic level of the prior art. More specifically, the method of diagnosing GERD according to the present invention by treating the endoscopic and symptomatic level as unit allows the administration of the most appropriate therapy in order to increase the probability to achieve a complete GERD remission. Thereby, the term “complete GERD remission” implies both, endoscopic healing of mucosal breaks and symptom relief in all situations, such that physical and psychical integrity is achieved. Moreover, when the method of monitoring the disease status according to the present invention is used, a reduced proportion of relapse rates can be expected.

### DETAILED DESCRIPTION OF THE INVENTION

In the following, the various aspects of the present invention will be described in more detail.

Preferred embodiments of the present invention are subject of the dependent claims.

In the method of diagnosing GERD according to the invention, the endoscopic status and the symptomatic status are individually graded by means of grading parameters, which are then combined into a single parameter.

As used herein, the term “GERD” is intended to mean both, erosive esophagitis and enGERD.

The term “grading” as used herein means assigning a specific parameter to the endoscopic and symptomatic status, of the human subject or patient, which assignment is each correlated to the specific findings reflecting the endoscopic and symptomatic status. Preferably, the grading is effected by selecting a grading parameter for the endoscopic status from a first scale and the grading parameter for the symptomatic status from a second scale. The scale reflecting the endoscopic, and the symptomatic status is each not specifically limited, as long as each scale represents the severity of the endoscopic finding and of the symptoms, respectively. The grading parameters may be descriptive in nature, i.e. verbalize the corresponding endoscopic and symptomatic status. Moreover, the parameters may be signs or symbols. According to a preferred embodiment, the grading parameters are alphabetic or numerical characters.

The single parameter obtained by combining the above individual grading parameters may be a juxtaposition of these. In the case that the individual grading parameters are numerical and/or alphabetic characters, this may afford a single parameter which is a two-digit parameter. According to another preferred embodiment, the single parameter is a numerical value obtained by subjecting the grading parameters to an arithmetic calculation. According to this embodiment, the individual grading parameters are advantageously numerical characters, i.e. numbers. Using as the single parameter according to the invention a numerical value is especially suited for statistical evaluations of the endoscopic and symptomatic status of a large number of human subjects suspected to have GERD, for example in a clinical study.

According to a preferred embodiment, the grading parameter for the endoscopic status is based on the so-called LA (Los Angeles) classification described in L. R. Lundell, Gut 1999, 45, 172-180, which classification has been adapted by P. J. Kahrilas in Aliment Pharmacol Ther. 2000; 14 (10): 1249-58. The disclosures of these documents are incorporated by reference into the present application in their entirety.

The grading parameter for the endoscopic status may be determined by subjecting the GERD patient or the individual suspected to have GERD to endoscopy to observe the occurrence of visible mucosal breaks, i.e. lesions. To determine the endoscopic grading parameter, the result of the endoscopic examination may be assigned to the parameters N to D, as follows:
N No mucosal breaks observed;
A One (or more) mucosal break no longer than 5 mm, that does not extend between the tops of two mucosal folds;
B One (or more) mucosal break more than 5 mm, that does not extend between the tops of two mucosal folds;
C One (or more) mucosal break that is continuous between the tops of two or more mucosal folds but which involves less than 75% of the esophageal circumference;
D One (or more) mucosal break which involves at least 75% of the esophageal circumference.

For the purpose of the present application, the above scale for grading the endoscopic status is designated as the adapted LA-classification.

In the alternative, the above range of N to D indicating the severity of the endoscopic status may be rescaled to the numerical range of from 0 to 4, as follows.
0 No mucosal breaks observed;
1 One (or more) mucosal break no longer than 5 mm, that does not extend between the tops of two mucosal folds;
   2 One (or more) mucosal break more than 5 mm, that does not extend between the tops of two mucosal folds;
3 One (or more) mucosal break that is continuous between the tops of two or more mucosal folds but which involves less than 75% of the esophageal circumference;
4 One (or more) mucosal break which involves at least 75% of the esophageal circumference.

This scale is referred to as the modified LA-classification, hereinafter.

There is a number of scales of symptomatic grading parameters described in the prior art. In most cases, the symptom severity is classified in the four categories none, mild, moderate and severe. Examples of such scales of parameters, which may be also used in the present invention to grade the symptomatic status are given in the following table.

| Grading | None | Mild | Moderate | Severe |
|---|---|---|---|---|
| Galmiche | complete | awareness | discomfort | incapacitating |
| et al. [1] | absence | but easily | sufficient to | with inability |
| | | tolerated | interference | to perform |
| | | | with normal | normal |
| | | | activities | activities |
| | | | such as work | |
| | | | and sleep | |
| Bardhan et | no def- | symptoms | clearly | overwhelming |
| al. [2] | inition | were hardly | noticeable | discomfort, |
| | | perceived, | symptoms, but | urgent desire |
| | | only slight | to tolerable | for immediate |
| | | impairment | without | relief |
| | | of general | immediate | |
| | | well being | relief | |
| Hattlebakk | Not | awareness of | interference | inability to |
| et al. [3] | included | symptom but | with normal | perform normal |
| | | easily | activities | activities |
| | | tolerated | | |
| van Zyl et | no def- | barely | clearly | overwhelming |
| al. [4] | inition | noticeable | noticeable | discomfort, |
| | | | but tolerable | urgent desire |
| | | | without | for immediate |
| | | | immediate | relief |
| | | | relief | |
| Venables | 0 | 1 = aware of | 2 = dis- | 3 = incapac- |
| et al. [5] | | symptom but | comfort | itating with |
| | | easily | sufficient to | inability to |
| | | tolerated | interfere | perform normal |
| | | | with normal | activities |
| | | | activities | |

[1] Aliment Pharmacol Ther. 1997; 11(4): 765-73.
[2] Aliment Pharmacol Ther. 2001; 15(10): 1585-91.
[3] BMJ. 1999 Aug. 28; 319(7209): 550-3.
[4] Eur J Gastroneterol Hepatol. 2000; 12(2): 197-202.
[5] Scand J Gastroenterol. 1997; 32(10): 965-73.

According to a preferred embodiment, the grading parameter for the symptomatic status for use in the method according to the invention is selected from a scale ranging from 0 to 4. Thereby, the numerical characters/numbers 0 to 4 are assigned to the symptomatic status which reflects the symptom burden, as follows:
0: no disease value
1: Minor symptoms
2: Tolerable symptoms
3: Troublesome symptoms
4: Intense symptoms.

The grading parameter 0 describes the “no disease value”. The absence of disease specific complaints, no impairment of well-being, and rare symptoms not worth mentioning may be assigned to the corresponding symptomatic status.

Turning to the grading parameter 1 designating the presence of minor symptoms, this may imply that there are rare symptoms with no need of medication. The impairment of well-being may be described as hardly noticeable. The person having a symptomatic status graded 1 is aware of the symptoms but will tolerate those easily.

In the case of tolerable symptoms according to a grading parameter of 2, these symptoms do not interfere with daily activities. Medications are used occasionally, and a dietary restriction is unnecessary. The person concerned experiences a marked discomfort but finds this tolerable without immediate relief.

A symptomatic status corresponding to the grading parameter of 3, i.e. indicating troublesome symptoms, will interfere with daily activities. Chronic medications, as well as dietary restrictions, are imperative. The affected individual feels continuous discomfort and a clear desire for relief.

Intense symptoms designated by the grading parameter 4 will incapacitate and prevent the concerned individual from daily activities. There is a partial relief on medication. Weight loss is often observed. The person will be unable to perform normal activities and experience an overwhelming discomfort with urgent desire for immediate relief.

The grading parameters for the symptomatic status may be determined by way of assessment of an investigator, e.g., an interview of the human subject suspected to have GERD. For example, this interview may be carried out by the investigator e.g the attending doctor asking the person suspected to have or having GERD for the presence of leading GERD-related symptoms.

The three leading GERD-related symptoms described in literature are: (1) heartburn, (2) acid regurgitation and (3) pain on swallowing (S. Kaspari et al., Digestion 2001; 63(3): 163-70; T. Korner et al., Digestion 2003; 67 (1-2): 6-13, J. H. van Zyl et al., Eur. J. Gastroenterol Hepatol 2000; 12(2): 197-202). Other associated GERD-related symptoms which may also be assessed during the interview are e.g., burning in the throat, dysphagia, pressure in the upper and lower abdomen, sore throat, nausea or vomiting. Heartburn is regarded as the most common GERD symptom occurring in at least 75% of patients (J. Dent et al., Gut 1999; 44 (supplement 2):S1-S16). Relevant GERD symptoms are also described by P. Bytzer in Gut 2004; 53 (Suppl. IV): iv28-iv34.

However, for the sake of comparability and as patients tend to underestimate their symptoms in an interview, it is preferred to rely on a self-assessment of the patients to evaluate their symptomatic status (see also E. McColl, Gut 2004; 53 (Suppl. IV): iv49-iv54). Questionnaires answered by the persons suspected to have GERD turned out to be a reliable objective tool to accomplish this. A large number of questionnaires has been described in the prior art, an overview of which is given in V. Stanghellini et al., Aliment Pharmacol Ther. 2004; 19 (5): 463-479. However, most of these questionnaires have the drawback that the symptom severity is assessed only at the end of treatment. Therefore, the questionnaire which, upon evaluation, affords the grading parameter for the symptomatic status is preferably the so-called Reflux Questionnaire (ReQuest™). This questionnaire is a validated, reliable, high responsive, evaluative instrument which is described in KD. Bardhan et al. (2004): Evaluation of GERD symptoms during therapy. Part I: Development of the new GERD questionnaire ReQuest™. Digestion 69: 229-237 and by H. Mönnikes et al. (2004): Evaluation of GERD symptoms during therapy. Part II: Psychometric evaluation and validation of the new GERD questionnaire ReQuest™. Digestion 69: 238-244

The Reflux Questionnaire (ReQuest™) represents a self-assessed questionnaire on a daily basis. The ReQuest™ comprises seven dimensions, namely general well-being, acid complaints, upper abdominal/stomach complaints, lower abdominal/digestive complaints, nausea, sleep disturbances, and other complaints. The short version of ReQuest™ involves for each of the above dimensions a leading question for frequency (7-point Likert scale) and intensity (degree of impairment with 100 mm visual analogue scale) with the exception of the dimension of general well-being which is tested for intensity only. The long version also includes 67 symptom descriptions assigned to the seven dimensions to be marked by the patient. The result of evaluating the ReQuest™ questionnaire may be converted to the grading parameter for the symptomatic status according to the invention.

ReQuest™ may be subdivided into two subscales comprising the following dimensions:

1) ReQuest™-GI: acid complaints, upper abdominal/stomach complaints, lower abdominal/digestive complaints, and nausea.

2) ReQuest™-WSO: general well-being, sleep disturbances, and other complaints.

According to a preferred embodiment, the ReQuest™-GI subscale is used to obtain the grading parameter for assessing the symptomatic status of the person concerned.

The dependency of the general well-being dimension and the ReQuest-GI subscale was examined using different models including linear regression and log linear regression. Using residual analyses it could be shown that the log linear model fits as the best (p<0.0001, R-Square=0.6665). From this model fitting the following formula could be established: Log(ReQuest-GI)=−0.26647+0.79951*(General Well Being). Hence these subscales have been shown to correlate. This assumption is also supported by E. J. Irvine, Gut 2004; 53 (Suppl. IV):iv35-iv39.

Using a face valid linear subdivision of the general well-being dimension into 4 descriptive grading parameters ranging from “no disease value” to “troublesome” the corresponding values of the ReQuest-GI subscale could be calculated using the above formula resulting in

No disease value: 0-1.70

Minor: 1.71-3.79

Tolerable: 3.80-8.43

Troublesome: 8.44-18.75

A patient having values above 18.76 is defined as “Intense”. The parameter range goes from 0 to 36.

On the basis of the above calculations, the parameters from the range of 0 to 36 may be transformed to the grading parameters 0 to 4 for the symptomatic status, according to a preferred embodiment of the invention, as follows:

| Grading Parameter for | Parameters of ReQuest ™ | Description of |
|---|---|---|
| symptomatic status | Gastrointestinal Subscale | symptom burden |
| 0 | 0 to 1.70 | No disease value |
| 1 | 1.71 to 3.79 | Minor |
| 2 | 3.80 to 8.43 | Tolerable |
| 3 | 8.44 to 18.75 | Troublesome |
| 4 | 18.76 to 36.00 | Intense |

The evaluation of the questionnaire to obtain the grading parameter the symptomatic status may be carried out manually but may additionally involve the use of a processing device. Hence, the 100 mm visual analogue scales and the 7-point Likert scale involved in the ReQuest™ may be measured manually and the obtained values be entered into the processing device to carry out the further evaluation. According to the present invention, the processing device is preferably a computer. Reading the above analogue scales and 7-point Likert scale may also be carried out with the aid of a scanner. Hence, the evaluation of the questionnaire may be carried out fully automated by way of a processing device such as a computer.

The processing device for diagnosing GERD in a human subject suspected to have GERD generally comprises means for grading the endoscopic status and the symptomatic status of the human subject with individual grading parameters, and means for combining said grading parameters into a single parameter.

In the case that the grading parameter for the symptomatic status is obtained by evaluating a questionnaire, the processing device comprises means for evaluating a questionnaire answered by a human subject suspected to have GERD to afford a grading parameter for the symptomatic status, and means for combining this with a grading parameter for the endoscopic status to afford a single parameter. More specifically, the grading parameter designating the endoscopic status may be entered by a first input unit of the processing device and the data contained in a questionnaire on the symptomatic status may be entered by a second input unit. The first and the second input unit are preferably the same and may be a keyboard. The second input unit may also comprise a scanner to input data from the questionnaire, such as from the above visual and 7-point Likert scales. The above data entered into the computer by the first and second input unit may then be converted to a single parameter reflecting the GERD disease state of the human subject under examination, in an evaluation unit, and the single parameter may then be indicated on an output unit, such as a screen or a printer.

Preferably, the above grading parameters designating the endoscopic and the symptomatic status, respectively, are combined into a single parameter as will be explained, hereinafter.

According a first preferred embodiment, the endoscopic status is graded N to D according to the adapted LA-classification as explained above and the scale of the grading parameter for the symptomatic status, which is advantageously obtained by evaluating the gastrointestinal subscale of ReQuest™, ranges from 0 to 4, after transformation of the ReQuest™ evaluation results. The grading parameter designating the symptomatic status is advantageously obtained by evaluating the gastrointestinal subscale of ReQuest™ but may also be obtained through an investigator's assessment of the leading GERD-related symptoms. The thus-obtained grading parameters for the endoscopic and symptomatic status, respectively, are then combined by juxtaposition into a 2-digit parameter, as follows.

| | |
|---|---|
| Symptoms: ReQuest ™ | Adapted LA-Classification |

| | | | | | |
|---|---|---|---|---|---|
| symptom classification | N | A | B | C | D |

| | | | | | | |
|---|---|---|---|---|---|---|
| No disease value | 0 | 0N | 0A | 0B | 0C | 0D |
| Minor | 1 | 1N | 1A | 1B | 1C | 1D |
| Tolerable | 2 | 2N | 2A | 2B | 2C | 2D |
| Troublesome | 3 | 3N | 3A | 3B | 3C | 3D |
| Intense | 4 | 4N | 4A | 4B | 4C | 4D |

The above 2-digit parameter has been designated as the ReQuest™/LA-classification” by the present inventors. As evident from the above, this parameter goes from 0N, corresponding to the complete GERD remission, to 4D, designating the most severe state of GERD.

According to an alternative preferred embodiment, the grading parameter for the endoscopic status corresponding to the modified LA-classification may be combined with the above symptomatic grading parameter which may be derived from ReQuest™ but also from an investigator's assessment as explained above to afford, by juxtaposition of the corresponding grading parameters another 2-digit parameter consisting of two numbers, as follows.

| | |
|---|---|
| Symptoms: ReQuest ™ | Modified LA-Classification |

| | | | | | |
|---|---|---|---|---|---|
| symptom classification | 0 | 1 | 2 | 3 | 4 |

| | | | | | | |
|---|---|---|---|---|---|---|
| No disease value | 0 | 0\|0 | 0\|1 | 0\|2 | 0\|3 | 0\|4 |
| Minor | 1 | 1\|0 | 1\|1 | 1\|2 | 1\|3 | 1\|4 |
| Tolerable | 2 | 2\|0 | 2\|1 | 2\|2 | 2\|3 | 2\|4 |
| Troublesome | 3 | 3\|0 | 3\|1 | 3\|2 | 3\|3 | 3\|4 |
| Intense | 4 | 4\|0 | 4\|1 | 4\|2 | 4\|3 | 4\|4 |

The single parameter as shown in the above table has been called the “Index” by the present inventors. In this case, the complete GERD remission status corresponds to 0|0 and the most severe form of GERD to 4|4.

Moreover, the numerical characters/numbers 0 to 4 designating the endoscopic and symptomatic status, respectively, may be combined by way of an arithmetic calculation affording a single number constituting a single parameter according to the invention. While not being limited in kind, the arithmetic calculation may involve some sort of weighting of the parameters for the endoscopic and the symptomatic status. It is evident from the above that the method of diagnosing GERD according to the present invention allows to assess more precisely whether an individual suffers from GERD than possible by the separate and independent usage of endoscopic and symptomatic aspects of diagnosing GERD as applied in the prior art. On this basis, it can be decided more reliably whether a GERD treatment is necessary, and whether an ongoing treatment should be continued. Using the two-digit parameters according to the above preferred embodiments of the invention, an individual suspected to have GERD would be regarded as in complete GERD remission if this were put into category 00 or 0|0. If the diagnosis of GERD according to the method of the invention afforded a single parameter different from 00 or 0|0 in these cases, a treatment of GERD would be initiated or continued. Such a treatment comprises the administration of a medicament having an ameliorative effect for GERD. Typical medicaments having an ameliorative effect for GERD comprise active ingredients selected from the class of proton pump inhibitors (PPI's), H₂ receptor antagonists, and acid pump antagonists (APA). Suitable proton pump inhibitors are omeprazole, rabeprazole, esomeprazole, lansoprazole, tenatoprazole, pantoprazole and/or (S)-pantoprazole and pharmaceutically acceptable salts of these compounds. Preferably, the medicament having an ameliorative effect for GERD comprises pantoprazole or pharmaceutically acceptable salts thereof as an active ingredient. According to a particularly preferred embodiment, the medicament comprises, as an active ingredient, sodium pantoprazole or magnesium pantoprazole. An example of an H₂ receptor antagonist is ranitidine. The acid pump antagonist may be soraprazan. Especially in cases, where the GERD patient to be treated has no endoscopically detectable signs of GERD, the treatment of GERD may also involve the administration of antacids. Thereby, the patient status “no endoscopically detectable signs of GERD” may be defined by the adapted or modified LA-classification being N. Once the investigator, e.g. the attending doctor has decided on the appropriate therapy to be applied, which decision is favorably made by means of the method of diagnosing GERD according to the invention, the single parameter as defined herein may be used to monitor the course of the treatment success of treating GERD, e.g. by administration of the above medicaments.

When monitoring the course of the treatment success, i.e. GERD remission, endoscopy to grade the endoscopic status of the patient is preferably carried out only occasionally, as this is an unpleasant experience for the patient. In clinical studies, for example, endoscopy is typically carried out every four weeks. In contrast, the symptomatic status may be graded on the basis of a questionnare which is answered daily.

The present invention also relates to a method of treating GERD in a human patient in need thereof, wherein this need is defined by a single parameter obtainable by combination of an individual grading parameter for the endoscopic status and an individual parameter for the symptomatic status of the human patient, which method comprises administering to the human patient a medicament having an ameliorative effect for GERD containing an active ingredient which may be selected from the class of proton pump inhibitors, H₂ receptor antagonists and acid pump antagonists. According to another aspect, the present invention is concerned with a method for treatment of a human patient who has been diagnosed as GERD-positive by a diagnostic method according to the invention, comprising administering to the human patient a medicament having an ameliorative effect for GERD as described above. According to a specific embodiment, the human patient diagnosed GERD-positive with a method according to the invention has no endoscopically detectable signs of GERD. Said human patient may further have only weak GERD symptoms.

In particular, the present application relates to a method for treatment of a human patient having an endoscopic GERD status of N according to the adapted or modified LA-classification as defined above, which method comprises administering to said human patient a medicament having an ameliorative effect for GERD such as described above. The human patient being subjected to the method of treatment according to the invention, apart from having an endoscopic GERD status of N according to the adapted or modified LA-classification, preferably has a symptomatic GERD status graded 1 or 2, more preferably graded 1. Differently stated, the present invention, according to a preferred embodiment relates to a method of treating GERD by administration of a medicament having an ameliorative effect for GERD to a patient who has been classified as 1N or 2N, preferably 1N according to the ReQuest™/LA-classification or who has an Index of 1|0 or 2|0, preferably of 1|0.

The single parameter according to the present invention constituting the first attempt to unify the endoscopic and symptomatic aspects of GERD is particularly sensitive and as such suitable to categorize human individuals having or being suspected to have GERD more precisely than previously possible. Therefore, human subjects may be diagnosed as GERD-positive which may previously have been regarded as GERD-negative based on only endoscopic assessment. This is likely to be the case for individuals, the endoscopic GERD status of which has been graded negative in the diagnostic method according to the invention. In particular, such persons, i.e. patients may be those, which have been graded N using the preferred grading parameter according to the present invention. Those patients in particular, which not only have an endoscopically negative GERD as explained above (i.e. enGERD) but which moreover have only weak GERD symptoms may be regarded as GERD positive using the diagnostic method of the invention whereas these persons would have been regarded as healthy based on the endoscopic assessment before the present invention was made. This especially applies to patients, the symptomatic GERD status of which has been graded 1 or 2 according to the above preferred scale of the symptomatic grading parameter ranging from 0 to 4, apart from the fact that these patients have no endoscopically detectable signs of GERD, i.e. have a grading parameter according to the adapted or modified LA-classification. More specifically, patients placed into category 1N or 2N according to the ReQuest™ LA-classification and/or those having an Index of 1|0 or 2|0 would be regarded as GERD-positive and the GERD therapy be initiated or continued accordingly until a complete GERD remission is achieved, whereas these patient groups may not have been treated or the treatment continued before the present invention was made. Furthermore, the relapse rates of GERD patients in complete remission can be expected to be lower when the method for diagnosing GERD according to the present invention is used, than previously possible.

The present invention also relates to a means for displaying the single parameter of the invention which comprises means to enter the individual grading parameters for the endoscopic and the symptomatic status and means for combining these grading parameters. Such a means may be a display wheel allowing the investigator, e.g. the attending doctor to enter the individual grading parameters by rotating discs of the wheel to specific positions and reading off the displayed single parameter obtained from the means for combining the individual grading parameters. Such a means for displaying the single parameters of the invention is particularly useful if the combination of the individual grading parameters to afford the single parameter involves subjecting the grading parameters to an arithmetic calculation. In these cases, the means for displaying may also be a calculator or a computer adjusted to combine the entered individual grading parameters.

1. A method of diagnosing GERD (gastroesophageal reflux disease) in a human subject suspected to have GERD which comprises grading the endoscopic status and the symptomatic status of the human subject with individual grading parameters, and combining said grading parameters into a single parameter.

2. The method according to claim 1, wherein the grading parameter for the endoscopic status is selected from a first scale, and the grading parameter for the symptomatic status is selected from a second scale.

3. The method according to claim 1, wherein the single parameter is a two-digit parameter.

4. The method according to claim 1, wherein the single parameter is a numerical value.

5. The method according to claim 4, wherein the numerical value is obtained by subjecting the grading parameter for the endoscopic status and the grading parameter for the symptomatic status to an arithmetic calculation.

6. The method according to claim 2, wherein the first scale of the grading parameter for the endoscopic status ranges from N to D.

7. The method according to claim 2, wherein the first scale of the grading parameter for the endoscopic status ranges from 0 to 4.

8. The method according to claim 2, wherein the second scale of the grading parameter for the symptomatic status ranges from 0 to 4.

9. The method according to claim 1, wherein the grading parameter for the symptomatic status is obtained from the result of an interview of the human subject.

10. The method according to claim 1, wherein the grading parameter for the symptomatic status is obtained from the evaluation of a questionnaire answered by the human subject.

11. The method according to claim 10, wherein the evaluation of the questionnaire is carried out by means of a processing device.

12. The method according to claim 10, wherein the questionnaire is the Reflux Questionnaire (ReQuest™).

13. A processing device for diagnosing GERD in a human subject suspected to have GERD comprising means for grading the endoscopic status and the symptomatic status of the human subject with individual grading parameters, and means for combining said grading parameters into a single parameter.

14. A method of treating GERD in a human patient who has been diagnosed as GERD-positive by a method according to claim 1, comprising administering a proton pump inhibitor to said patient.

15. The method according to claim 14, wherein the proton pump inhibitor is selected from the group consisting of omeprazole, rabeprazole, esomeprazole, lansoprazole, tenatoprazole, pantoprazole, (S)-pantoprazole and pharmaceutically acceptable salts thereof.

16. The method according to claim 14, wherein the proton pump inhibitor is pantoprazole or a pharmaceutically acceptable salt thereof.

17. A method of treating GERD in a human patient who has been diagnosed as GERD-positive by a method according to claim 1, comprising administering an H₂ receptor antagonist to said patient.

18. A method of treating GERD in a human patient who has been diagnosed as GERD-positive by a method according to claim 1, comprising administering an acid pump antagonist to said patient.

19. The method according to claim 14, wherein the human patient has no endoscopically detectable signs of GERD.

20. The method according to claim 14, wherein the human patient has an endoscopic GERD status which has been graded N or 0.

21. The method according to claim 19, wherein the human patient further has only weak GERD symptoms.

22. The method according to claim 19, wherein the symptomatic GERD status of the human patient has been graded 1 or 2.

23. A means for displaying the single parameter obtained in the method according to claim 1, comprising means to enter the individual grading parameters for the end.

24. The means according to claim 23 which is a display wheel.

25. The method according to claim 20, wherein the human patient further has only weak GERD symptoms.

26. The method according to claim 20, wherein the symptomatic GERD status of the human patient has been graded 1 or 2.
